(19) United States
(12) Patent Application Publication
(10) Pub. No.: US 2023/0040901 A1
(43) Pub. Date: Feb. 9, 2023

(54) PHARMACEUTICAL COMPOSITION FOR INHIBITING POSTOPERATIVE ADHESIONS

(71) Applicant: PlantTec Medical GmbH, Luneburg (DE)

(72) Inventors: Joachim LAAS, Bad Bevensen (DE); Kai-Oliver MUTZ, Hannover (DE)

(73) Assignee: PlantTec Medical GmbH, Luneburg (DE)

(21) Appl. No.: 17/941,487

(22) Filed: Sep. 9, 2022

Related U.S. Application Data
(63) Continuation-in-part of application No. 17/525,465, filed on Nov. 12, 2021.
Application No. 17/525,465 is a continuation of application No. 16/320,047, filed on Aug. 4, 2020, now abandoned.

(30) Foreign Application Priority Data
Jan. 4, 2018 (DE) 10 2018 000 009.6

(51) Int. Cl. A61K 31/718; A61P 17/02; A61K 31/194

(52) U.S. Cl. CPC A61K 31/718; A61P 17/02; A61K 31/194; A61K 9/06

(57) ABSTRACT

The pharmaceutical composition constituted by carboxymethylated starch and citric acid, a citrate, or mixtures thereof, prevents adhesion of injured mesothelial surfaces and the formation of adhesion bands between the mesothelia at the highest level. The composition in gel form acts as a matrix in which connective tissue healing of tissue defects is promoted.

### CROSS-REFERENCE TO RELATED APPLICATIONS

This application is a continuation-in-part of U.S. Ser. No. 17/525,465 filed on Nov. 12, 2021, which in turn is a continuation of U.S. Ser. No. 16/320,047 filed on Aug. 4, 2020 (now abandoned), which is a National Stage Entry of PCT/DE2019/100001 filed on Jan. 3, 2019, which claims priority to DE 10 2018 000 009.6 filed Jan. 4, 2018, each of which is incorporated herein by reference.

### FIELD OF THE INVENTION

The invention provides a method for inhibiting the formation of postoperative adhesions comprising administering to a subject in need thereof a pharmaceutical composition comprising or consisting of a carboxymethylated starch and sodium citrate. The invention also relates to a pharmaceutical composition comprising or consisting of a carboxymethylated starch and sodium citrate; and a method for producing said pharmaceutical composition.

### BACKGROUND ART

Organs and tissues in the body that are covered with a mesothelium (heart, lungs and intra-abdominal organs, parietal wall of the pericardium, thorax and abdominal cavity) are normally able to freely move and are separate from each other, even when their surfaces are in close contact. Articular cavities are covered with the synovium, which is essential there for the free movement of bones and joints. This also applies to the tendons and their tendon sheaths. After surgical interventions in the aforementioned body cavities or on the synovial structures, physiological wound healing starts and results in wound closure or covering of the wounds to protect them from infection. The processes that cause physiological wound healing are basically the same processes that lead to the formation of adhesions, which can cause a variety of problems. Adhesions may appear as slight accumulations that are easy to dissolve or as bands of fibrous, sometimes vascularized tissue (i.e. connective tissue containing blood vessels). They may even appear as complete adhesions, which abnormally fix the organs permanently. Adhesions may cause chronic pain, disorders of the abdominal viscera such as obstruction of the intestinal passage up to total obstruction (ileus) and thus lead to further surgical interventions or even death. Furthermore, adhesions in the area of the internal genital organs of women are the most frequent cause of secondary female infertility. Adhesions increase the risk during surgery due to bleeding during removal of the adhesions. Moreover, the areas removed of adhesions are hearths for the development of new adhesions. Adhesions in synovial structures may cause chronic pain and movement impairment. Medical complications aside, the treatment of adhesions is a significant socioeconomic problem. For this reason, a safe and low-risk prevention and/ or inhibition of adhesions is one of the main goals of modern surgery.

Surgeries are the most frequent cause for the formation of adhesions and they may be a serious complication. The predisposing factors include mechanical injuries of mesothelial (mesothelium-like such as, for example, synovial) surfaces or peritoneal ischemia due to manipulation and retraction of tissues during operation.

The incidence of postoperative adhesion formation is estimated today at 67% to 93%. At present, there is a variety of barriers on the market for reducing this problem. While the principle is invariably the physical separation of wound areas, the products differ considerably due to their application, structure and composition. There are barriers or rinse solutions produced from cellulose or polytetrafluorethylene, icodextrin, hyaluronic acid as well as carboxymethyl cellulose.

US 2002/0169144A1 discloses a pharmaceutical composition that comprises a cross-linked polyanionic polysaccharide, such as carboxymethyl amylose and a citrate buffer, the composition preventing the formation of surgical adhesions.

US 2004/0153040 A1 discloses a wound dressing comprising a layer of hydrophilic polymer foam and a hydrogel layer used to contact the surface and as an anti-adhesion ingredient, wherein the hydrogel layer may contain carboxymethylated starch, wherein the polymer foam may be cross-linked with citric acid and the wound bandage is used during a postoperative treatment.

WO 2016/098057 A1 discloses a chitosan hydrogel, which includes citric acid and carboxymethylated starch, the hydrogel being used to counteract postoperative adhesions.

DE 10 2013 211 316 A1 discloses a hemostatic agent comprising carboxymethylated starch, which is chemically cross-linked by citric acid.

WO 2016/100861 A1 discloses a hemostatic composition comprising cross-linked modified amylopectin, icodextrin or maltodextrin, which includes carboxymethylated starch, carboxymethyl maltodextrin or carboxymethyl icodextrin, as well as sodium citrate as a constituent of an aqueous diluent.

In addition to commercial products for reducing or inhibiting postoperative adhesions, other medicinal materials are also conceivable. The most promising solutions appear to be the use of modified starches as mechanical barriers, as well as the use of citrate for inhibiting the formation of fibrin. Hoffmann et al. (2009), for example, reported that products for hemostasis may also reduce postoperative adhesions. Starch-based products for hemostasis, in particular, significantly reduce the degree of adhesions as compared to other “conventional” topical products for hemostasis. With Arista™ AH (disclosed by the patent family U.S. Pat. No. 6,060,461), consisting of microporous starch granules, it is possible to achieve a maximum 50% reduction of postoperative adhesions in the rat model. WO2009/091549 discloses, in particular, a hemostatic material, which in this case is a modified starch having a water absorption capacity of no lower than the simple inherent weight, having a molecular weight of greater than 15,000 Daltons and having a starch granule diameter of 10 to 1,000 at least 95% of the starch granules having a diameter between 30 μm and 500 μm. This modified starch is biocompatible, absorbable and is degraded by amylases and carbohydrases. Methods with regard to application are disclosed, in which the hemostatic material is applied to tissue, for example, in the form of powder or as a gel and is used, inter alia, for the purpose of hemostasis and adhesion prophylaxis. In two publications, Pope et al. (1914 and 1916) report on a reduction of postoperative adhesions due to the use of citrate. It was shown in 60 experiments on rabbits that 2% to 4% citrate in water or saline solution resulted in fewer adhesions. This was confirmed in clinical application in 400 abdominal surgeries and it was shown that a clear improvement with respect to the development of adhesions was evident as result of citrate.

### SUMMARY OF THE INVENTION

Alternative solutions to the problem of postoperative adhesions are always desirable. The fact, however, that adhesions may be reduced to a never before seen degree by modified starch (synergistically improved compared to modified starch as a mechanical barrier or citrate for inhibiting fibrin formation, in each case individually), which contains citrate and citric acid (either from the manufacturing process or through admixture), applied to traumatized mesothelial (or mesothelium-like) surfaces (zones at risk for developing adhesions) and transformed from starch powder to a starch gel, is unique and not yet known.

The pharmaceutical composition according to the invention has an excellent utility for reducing and/or inhibiting postoperative adhesions and is suitable for reducing and/or inhibiting postoperative adhesions in an unprecedented way, since the pharmaceutical composition according to the invention combines the mechanical barrier effect of a modified starch gel on the one hand and the inhibitory effect of citrate on the polyfibrin — formation on the other hand synergistically .

The invention provides a pharmaceutical composition that utilizes a carboxymethylated starch together with 1 to 20 percent by weight citric acid and/or a citrate, such as a salt of citric acid, relative to the weight of the carboxymethylated starch, and a method for reducing and/or inhibiting postoperative adhesions and for treating of disorders associated with the surgery of mesenchymal organs or tissues, of organs in serous cavities or of epithelial tissue.

### BRIEF DESCRIPTION OF THE DRAWINGS

FIG. 1: Starch in powder form (left) and as a premixed gel (right). The starch powder had the composition of Medication 1 as described in Example 1.

FIG. 2: Microscopic image of the swollen powder particles/gel particles.

FIG. 3: Experimental steps for testing of efficiency of the pharmaceutical composition of the invention in adhesion inhibition.

FIG. 4: Macroscopic view of an untreated control animal. The cecum that has grown into the abdominal wall cannot be spared despite strong traction with the forceps.

FIG. 5: Histological section through the edge of the abdominal wall defect of an untreated control animal on Day 7 after OPAM (staining with hematoxylin and eosin). Normal cecal mucosa and submucosa (1), smooth musculature of the cecum (2), fibrous tissue (3) connecting cecum and abdominal wall, and skeletal muscle of abdominal wall (4). The edges of the transected striated musculature of the abdominal wall are closely fused with the muscularis of the cecum. There is no filling of the abdominal wall defect with a separate scar.

FIG. 6: Macroscopical view of the situs seven days after creation of abdominal wall defect and abrasion of cecum. For orientation the liver can be seen in the right upper abdomen. The abdominal wall defect laterally is filled with regenerated muscle and centrally with a white fibrous scar. There is no adhesion formation. The peritoneum has a shiny surface and appears unimpaired and normal. The thickness of the scar corresponds with the thickness of the normal abdominal wall.

FIG. 7: Histological sections of the cecum of an animal treated with the pharmaceutical composition of the present invention. Normal mucosa and submucosa (1), smooth muscle of the cecum (2), normal single layer peritoneal epithelium covering healed cecum (arrows).

FIG. 8: Histological section through the marginal zone of an abdominal wall defect in an animal treated with the pharmaceutical composition of the present invention. Fibrous scar tissue completely filling the abdominal wall defect (1), margins of the transected abdominal wall musculature at the border of the former defect (2), normal single layer normal peritoneal epithelium covering the healed defect (arrows).

FIG. 9: Horizontal section through the healed abdominal wall defect. The section shows (a) fibroblasts and early scar tissue in the center of the former lesion, (b) remnants of the hydrobead particles of the pharmaceutical composition of the present invention, (c) giant cells as a result of fusioned macrophages, (d) new formed small vessels and capillaries.

FIG. 10: Residual polysaccharide particle (diameter appr. 200 μm) surrounded by macrophages which also are degrading the particles from inside. Resolving groups of macrophages.

FIG. 11. Schematic sequence of a foreign body reaction.

### DETAILED DESCRIPTION OF THE INVENTION

The objective problem to be solved by the present invention is the provision of pharmaceutical composition with superior effects and a method for reducing and/or inhibiting postoperative adhesions and for treating of disorders associated with the surgery of mesenchymal organs or tissues, of organs in serous cavities or of epithelial tissue.

The pharmaceutical composition of the invention

The invention solves this problem by providing a pharmaceutical composition containing a carboxymethylated starch and 1 to 20 percent by weight citric acid and/or a salt of citric acid relative to the weight of the carboxymethylated starch.

The pharmaceutical composition of the invention comprises carboxymethylated starch in the form of powder particles, which preferably comprises a highly soluble citrate salt, wherein easy solubility is defined as a solubility of 100 g/l to 1000 g/l citrate in water at a temperature of 25° C. It is crucial for the pharmaceutical effect that free citrate ions are present in the pharmaceutical composition after the addition of an, preferably aqueous, liquid.

By adding an aqueous liquid, the carboxymethylated starch of the pharmaceutical composition forms a gel, which also comprises citrate ions from citric acid or from a highly soluble citrate salt. The gel acts as a barrier during the healing process of mesenchymal organs or tissues, of organs in serous cavities or of epithelial tissue after a surgery by mechanically preventing contacts between organ or tissue surfaces. The dissolved citrate inhibits the formation of polyfibrin during blood coagulation. Polyfibrin is a basic building block of adhesion formation.

The citric acid and/or the citrate salt may be included in the pharmaceutical composition in an amount of 1% to 20% of the total weight of the carboxymethylated starch. The content of citric acid and/or a salt of citric acid relative to the weight of the carboxymethylated starch is preferably 1 to 10 percent by weight, more preferably 1 to 8 percent by weight, most preferably 2 to 5 percent by weight.

In a preferred embodiment, the pharmaceutical composition of the invention comprises a carboxymethylated starch and 1 to 20 percent by weight of a salt of citric acid relative to the weight of the carboxymethylated starch, wherein said salt of citric acid has a solubility in water of greater than 100 g/l, preferably greater than 200 g/l, more preferably greater than 300 g/l, most preferably greater than 400 g/l, at a temperature of 25° C.

Said salt of citric acid is preferably selected from the group consisting of a sodium citrate, a lithium citrate, a potassium citrate, a calcium citrate and a magnesium citrate. Most preferably, said salt of citric acid is trisodium citrate, which is biocompatible and which has a solubility of 425 g/l at a temperature of 25° C.

Further most preferably, said citrate from citric acid or a citrate salt, especially trisodium citrate is located on the surface of the particles of the carboxymethylated starch.

The high solubility of the citrate salt and the location on the surface of the starch particles enable a fast dissolution of the citrate salt and a fast delivery of the dissolved citrate ions for unfolding of the pharmacological effect, namely the inhibition of the formation of polyfibrin during blood coagulation.

The pharmaceutical composition is advantageously present in the form of a gel. In addition, the pharmaceutical composition according to the invention advantageously further comprises water and/or at least one salt, for example and in particular sodium chloride or potassium chloride.

In a preferred embodiment, the pharmaceutical composition of the invention comprises carboxymethylated starch and 1 to 20 percent by weight citric acid and/or a salt of citric acid relative to the weight of the carboxymethylated starch, and, optionally water.

In a further preferred embodiment, the pharmaceutical composition of the invention comprises carboxymethylated starch and 1 to 20 percent by weight citric acid and/or a salt of citric acid relative to the weight of the carboxymethylated starch, water and/or at least one salt, for example and in particular sodium chloride or potassium chloride.

In practice, it has proven beneficial for the carboxymethylated starch to have a molecular weight of 500,000 daltons to 11,000,000 daltons, and/or for the starch particles, the carboxymethylated starch have a particle diameter in the range of 30 μm to 100 μm.

Accordingly, the invention provides a pharmaceutical composition comprising or consisting of carboxymethylated starch and 1 to 20 percent by weight trisodium citrate relative to the weight of the carboxymethylated starch, and optionally water, wherein said carboxymethylated starch has a molecular weight in the range of 500,000 daltons to 11,000,000 daltons, and/or wherein the starch particles of the carboxymethylated starch particles have a particle diameter of 30 microns to 100 microns.

Preferably, said carboxymethylated starch has a molecular weight in the range of 750,000 daltons to 10,000,000 daltons or 1,000,000 daltons to 9,000,000 daltons.

More preferably, said carboxymethylated starch has a molecular weight in the range of 2,000,000 daltons to 8,000,000 daltons or 3,000,000 daltons to 7,000,000 daltons.

Most preferably, said carboxymethylated starch has a molecular weight in the range of 4,000,000 daltons to 6,000,000 daltons.

The carboxymethylated starch of the pharmaceutical composition of the invention has the following, further preferred characteristics: It is a crosslinked, carboxymethylated starch containing approximately 20 wt % amylose and approximately 80 wt % amylopectin in a relatively constant ratio. The amylose is rather amorphous, and the amylopectin represents the crystalline portion of the starch. The modified starch is present as a salt-starch glycolate, and is generally the sodium salt of a carboxymethyl ether of the starch.

A starch particle of the carboxymethylated starch is made up of about 4.5×10¹⁰ to 2.3×10¹² amylose molecules and 5.6×10⁷ to 1.3 ×10¹⁰ amylopectin molecules. The degree of crosslinking of the carboxymethylated is preferably in the range of 25% to 45%.

The carboxymethylated starch may further contain at least one salt, which is selected from the group consisting of sodium chloride and potassium chloride, preferably sodium chloride, in an amount between 0 weight % and 10 weight %, preferably between 2 weight % and 5 weight % calculated on the basis of the dry mass of the carboxymethylated starch.

After addition of water, or when the pharmaceutical composition is in gel form, the pH value of the solution or gel is in the range of 3.0 and 7.5, preferably from 5.0 to 7.5, most preferably from 6.5 to 7.5.

The carboxymethylated starch is present as a white or almost white fine, free-flowing powder. The powder is very hygroscopic, and is practically insoluble in methylene chloride. It forms a translucent suspension in water.

The powder is made up of irregularly shaped, oval or pear-shaped particles in a size range from 30 to 100 μm, or 10 to 35 μm with rounding.

The starch particles have typically a surface of approximately 0.2 m²/g. Occasionally occurring clusters of particles are made up of two to four particles. The particles have an eccentric hilus and clearly visible concentric grooves. The particles exhibit a distinct black cross on the hilus, between intersecting Nicol prisms. Small crystals are discernible at the surface of the particles. The particles exhibit significant swelling, up to approximately 40 times their own weight, upon contact with water or salt solutions.

The carboxymethylated starch comprised in the pharmaceutical composition of the invention can be produced with the following method:

The synthesis of the carboxymethylated starch generally takes place in three steps, in each case as a suspension, wherein step 1 comprises crosslinking of the starch, step 2 comprises carboxymethylation of the starch, and step 3 is a neutralization step.

The crosslinking-step uses hydroxyl groups attached to starch (present in the basic glucose building blocks of the starch). The reaction of these hydroxyl groups with multifunctional reagents results in crosslinked starches. In a granule of starch, many chains are found in closer proximity, and such reactions not only take place between single chains, but it links side by side chains as well. These crosslinking agents form either ether or ester inter-molecular linkages between hydroxyl groups on starch molecules A small amount of multifunctional reagent is enough to interconnect starch molecules by crosslinking reactions. Methods for crosslinking of starches are generally known to the person skilled in the art. An overview of starch crosslinking is e.g. given in Shah, Nimish & Mewada, Rajubhai & Mehta, Tejal. (2016). Crosslinking of starch and its effect on viscosity behavior. Reviews in Chemical Engineering. 32. 10.1515/revce-2015-0047, which is incorporated herein in entirety.

In the present invention, the crosslinking step 1 of the starch is preferably performed with Na₃P₃O₉ (sodium trimetaphosphate).

Since the reactions take place in suspension, the substitution is inhomogeneous. Outer areas of the starch are more greatly affected by the modifications than the core areas of the starch structures. The reactions are influenced by diffusion within the starch structure and accessibility to the starch structure. Amorphous areas are more easily accessible than crystalline areas, which are less strongly modified. From a chemical standpoint, both reactions are nonspecific, and, provided that diffusion inhibition or steric hindrance is present, have no preferred reaction pattern for the individual hydroxyl groups of a glucose unit. Theoretically, the degree of substitution is 3 (with substitution of all three OH groups of a glucose unit as the monomeric building block of the starch). The actual degree of substitution appears to be in the range of about 0.2 to 0.4; i.e., one OH group is substituted in approximately every fourth glucose unit (not taking crosslinking into account). This degree of substitution results from the values for the bound salt.

After steps 1 and 2 have been performed, neutralization is carried out as step 3, using an acid. Preferably, said acid is selected from the group consisting of succinic acid, phosphoric acid, hydrochloric acid, sulfuric acid, and citric acid. The selection of the acid has an effect on inorganic and organic substances that can be detected as residues in the modified starch. Most preferred according to the invention is citric acid.

Accordingly, the pharmaceutical composition of the invention may further comprise an organic or inorganic salt resulting from the neutralization step, wherein said organic or inorganic salt is selected from the group consisting of succinate, phosphate, chloride, sulphate, and citrate.

To reach the content of 1 to 20 percent by weight of citric acid and/or a citrate salt relating to the weight of the carboxymethylated starch, citric acid or a citrate salt is added in the respective amount to the carboxymethylated starch in suspension and the components are intimately mixed.

In a preferred embodiment, when the pharmaceutical composition is in gel form, the invention therefore provides an aqueous gel comprising crosslinked carboxymethylated starch, citrate ions and water, wherein the citrate ions are present in a concentration in the range of 1 mmole/liter to 500 mmoles /liter of the water, and wherein the crosslinked carboxymethylated starch has a degree of substitution in the range of 0.2 to 0.4 and a degree of crosslinking in the range of 25% to 45%.

Method of the Invention and Mode of Administration to a Subject

The invention further provides a method for reducing and/or preventing and/or inhibiting postoperative adhesions, said method comprising administering to a subject in need thereof a pharmaceutical composition as described hereinabove.

In the simplest form of the method according to the invention the pharmaceutical composition is administered in powder form directly after a surgery and directly on a postoperative area or site to form a gel covering the postoperative area or site, or is mixed with an aqueous liquid and optionally salts to form a gel, which is then administered in gel form on the postoperative area or site. When administered in powder form directly on the postoperative area or site, a gel is formed in situ due to the presence of residual body fluids at the postoperative area or site, or by drizzling the powder after administration on the postoperative area or site with an aqueous liquid.

More specifically, the method of the invention comprises the following steps:
i) Providing the pharmaceutical composition according to claim 1 as a powder;
ii) Administering the powder of step i) directly on an postoperative area or site in a subject;
iii) Forming a gel of carboxymethylated starch covering the postoperative area or site
   By residual body fluids present at the postoperative area or site; or
   By drizzling the powder after administration on the postoperative area or site with an aqueous liquid,
   Wherein said gel of carboxymethylated starch comprises citric acid and/or a dissolved salt of citric acid.

In an alternative embodiment, the method of the invention comprises the following steps:
i) Providing the pharmaceutical composition according to claim 1 as a powder;
ii) Premixing the powder of step i) with an aqueous liquid;
iii) Forming a gel of carboxymethylated starch, which comprises citric acid and/or a dissolved salt of citric acid;
iv) Administering the gel of step iii) on the postoperative area or site in a subject.

In a further alternative embodiment, the method of the invention comprises the following steps:
i) Providing a pharmaceutical composition which comprises a gel of carboxymethylated starch, wherein said gel of carboxymethylated starch comprises citric acid and/or a dissolved salt of citric acid;
ii) Administering the gel of step i) on the postoperative area or site in a subject.

The aqueous liquid is preferably selected from the group consisting of water, isotonic saline solution, or hypotonic, isotonic, or hypertonic saline solution as the liquid phase, which comprises one or more cations selected from the group consisting of sodium, potassium, ammonium, magnesium, calcium, iron(II), iron(III), aluminum; and/or further comprising one or more anions selected from the group consisting of fluoride, chloride, bromide, iodide, oxide, sulfide, carbonate, sulfate, phosphate, nitrate, chromate, permanganate, hexacyanoferrate(II). Ringer's solution, Ringer's acetate solution, and Ringer's lactate solution may also be used as the liquid phase.

Accordingly, the pharmaceutical composition, when in gel form, may further comprise one or more cations selected from the group consisting of sodium, potassium, ammonium, magnesium, calcium, iron(II), iron(III), and aluminum; and/or further comprising one or more anions selected from the group consisting of fluoride, chloride, bromide, iodide, oxide, sulfide, carbonate, sulfate, phosphate, nitrate, chromate, permanganate, hexacyanoferrate(II), acetate and lactate.

Most preferably, the aqueous liquid is water or isotonic saline.

In the methods of the invention as described hereinbefore, the aqueous liquid, most preferably water or isotonic saline, is suitably added in a volume of 1-40 ml per gram powder of the pharmaceutical composition.

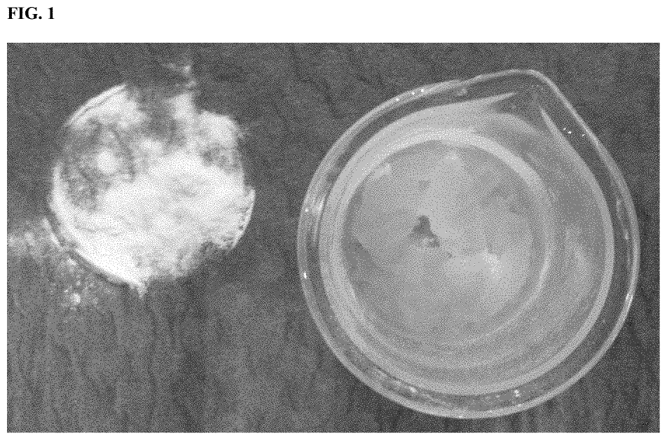

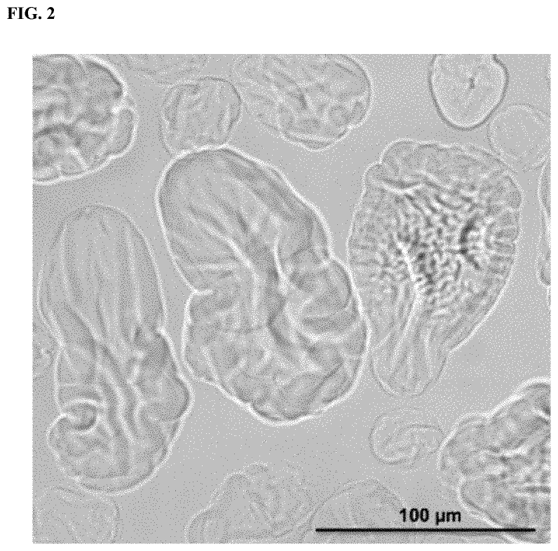

An amount of aqueous liquid is added to the starch powder, sufficient to form a translucent gel. FIG. 1 shows a starch of the invention in powder form (left) and as a premixed gel (right). FIG. 2 shows a microscopic image of the swollen powder particles/gel particles.

The subject is preferably a mammal. Accordingly, the pharmaceutical composition of the invention is applicable in veterinary and human medicine. Most preferably, the subject is a human and the pharmaceutical composition of the invention is applied in human medicine.

Mode of Action of the Pharmaceutical Composition of the Invention

The pharmaceutical composition of the invention comprises carboxymethylated and cross-linked starch, 1% to 20% percent by weight of which is citrate, as detailed hereinabove.

This combination offers never before seen positive results for reducing and/or inhibiting postoperative adhesions. This applies, in particular, to the reduction or prevention or inhibition of postoperative adhesions, in particular, traumatized mesothelial (or mesothelium-like) surfaces (zones at risk for developing adhesions) such as, for example, the pleura in the chest cavity, peritoneum in the belly and pelvic cavity or pericardium serosum in the pericardial cavity, as well as synovial structures of bones, joints, tendons or nerves.

Accordingly, the invention provides a method, wherein said postoperative adhesions are reduced or prevented on mesothelial or mesothelium-like surfaces selected from the group consisting of mesothelial surfaces of the peritoneum, pericardium, pleura, synovium and tendon sheaths.

Advantageously, the method of the invention further comprises the treatment of a disorder associated with a surgery of mesenchymal organs or tissues, of organs in serous cavities or of epithelial tissue.

Said disorder associated with a surgery of mesenchymal organs or tissues is preferably selected from the group consisting of impaired wound healing, epithelial defects and hemorrhage.

Said mesenchymal organs or tissues are preferably selected from the group consisting loose connective tissue, tight connective tissue, reticular connective tissue, bones, cartilage, smooth muscles, heart muscle, kidneys, adrenal cortex, hematopoietic system, blood vessels and lymphatic vessels.

The never before seen positive properties of the pharmaceutical composition of the invention for reducing and/or inhibiting postoperative adhesions is assumed to be based on the following mechanisms:

An efficient tissue separation during wound healing is guaranteed by the pharmaceutical composition of the invention, applied in gel form or transformed from powder into gel form, on traumatized mesothelial (or mesothelium-like) surfaces (zones at risk for developing adhesions). The application of the pharmaceutical composition of the invention in gel form or powder form or, for example, as a film, leads to the formation of a complete, effective and reliable barrier between organ and/or tissue surfaces and reaches every corner of the traumatized area after a surgery. Since carboxymethylated and cross-linked starch and citrate are absorbed within a few days, there is no need for a second surgery to remove the mechanical barrier. This makes it possible to prevent adhesions because the traumatized areas are covered with the starch-gel barrier and are thus physically separated from adjacent or other surfaces. The effect is synergistically reinforced by the effect of citrate, which inhibits fibrin formation, in particular polyfibrin formation.

Highly soluble salts of citric acid are used in medicine to prevent the coagulation of blood, e.g. donated blood (so-called citrated blood). Blood coagulation requires calcium ions as essential co-factors for blood clotting. Calcium ions are present in the blood plasma in a concentration of 46 to 54 mg/l. In the presence of citrate, the calcium ions form a very poorly soluble salt with the citrate (very poor solubility is defined here as a solubility of 0.1 to 1 g/l in water at 25° C.). The solubility of calcium citrate with 0.85 g/l is in this range). This salt precipitates immediately, so that free calcium ions are no longer available as a cofactor for blood clotting. This in turn inhibits the formation of polyfibrin. The end product of blood clotting, polyfibrin, is a basic building block of adhesion formation between the organs and tissues discussed hereinabove.

Citrate forms insoluble chelate complexes with calcium, as a result of which a formation and stabilization of fibrin polymerization sites in all subsequent stages of fibrin polymerization is achieved. Citrate limits the deposition of fibrin through the binding of calcium ions, in order in this way to render the subsequent formation of adhesions impossible. Additionally calcium ions are also co-factors for other steps of the coagulation cascade before the formation of fibrin: they are part of the prothrombinase complex and the extrinsic tenase complex, both of which would not function without it and are responsible for platelet activation as well as the activation of several coagulation factors. As a result, the pharmaceutical composition of the invention offers two different antiadhesive effects, physical barrier and fibrin polymerization inhibition.

Advantageously, in the pharmaceutical composition of the present invention, citric acid and/or a citrate salt, such as trisodium citrate, is located on the surface of the particles of the carboxymethylated starch. Trisodium citrate is highly soluble, i.e. it dissolves immediately in the presence of water or another aqueous liquid. The dissolved citrate is immediately available, which, together with calcium ions, immediately forms the very poorly soluble calcium citrate. The calcium citrate precipitates immediately and is no longer available for the deposition of fibrin.

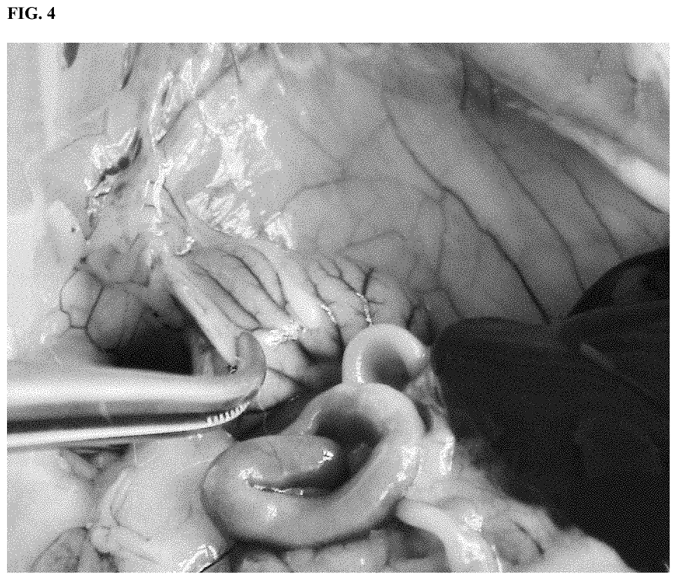

As the result of administering the pharmaceutical composition of the invention, a gel is acting locally on the postoperative area or site. The presence of the swollen starch powder particles in starch gel is associated with an increased invasion of cellular components of early healing. Activated macrophages come into contact with the surfaces of the swollen starch particles quite rapidly, forming a framework, followed by other cellular components of early healing (see FIG. 4). Although the starch framework/matrix is degraded within days, an increased number of cellular components remain in the healing area, resulting in improved wound healing, epithelial defects and hemorrhage, wherein in particular an increased number of macrophages for the macrophagocytosis of bacteria, apoptopic cells, necrotic cells, cells with foreign protein, and polysaccharide surfaces are observed in the anastomotic area. In addition, an increased number of fibroblasts and other cellular components of wound healing, epithelial defects and hemorrhage are also observed. The presence of cells for early healing and the subsequent wound healing processes are accelerated by these factors.

Histological analyses in an area have shown improved submesothelial healing after one week due to application of the above-mentioned starch gel, i.e., in a form of the pharmaceutical composition according to the invention showing remnants (residues) of hydrobeads, macrophages and giant cells, fibroblasts, and emerging connective tissue.

The improvement in healing results from the presence of the pharmaceutical composition according to the invention, which activates the early cellular healing processes, i.e., the attraction of macrophages, followed by the attraction of fibroblasts. The latter represent stabilizing factors for improving wound healing, epithelial defects and hemorrhage.

The subject matter according to the invention is explained in the following embodiment by way of two examples and in a nonlimiting manner.

Example 1: Adhesion Inhibition with Carboxymethylated Starch with Sodium Citrate—In Vivo Results

The adhesion inhibition efficacy of the pharmaceutical composition of the invention (carboxymethylated starch with 2.9—3.6% sodium citrate) was evaluated in an animal model using the established optimized peritoneal adhesion model (OPAM). The pharmaceutical composition of the invention used in the treatment group (further called “Medication 1”) had the following composition:
Medication 1
Chemical name: Starch, carboxymethyl ether, sodium salt, present as sodium glycolate
Appearance: white or almost white fine, free-flowing powder
Odour: neutral
Molecular weight: in the range of 500,000 daltons to 11,000,000 daltons
Particle size: in the range of 30 μm to 100 μm
Water content: 3.9% (w/w)
Sodium chloride: 4.0 (w/w)
Sodium glycolate: <2.0 (w/w)
Sodium citrate: 3.2% (w/w)
Degree of crosslinking: 34%
Crosslinking agent: Na₃P₃O₉ (sodium trimetaphosphate)

Thereby, an untreated control group where the animals only received sterile 0.9% saline solution was used as baseline. The pharmaceutical composition of the invention (Medication 1) was compared to two other interventions: carboxymethylated starch without sodium citrate (further called “Medication 2”) and sodium citrate without carboxymethylated starch (further called “Medication 3”) .

The carboxymethylated starch without sodium citrate (Medication 2) had the following composition:
Medication 2
Chemical name: Starch, carboxymethyl ether, sodium salt, present as sodium glycolate
Appearance: white or almost white fine, free-flowing powder
Odour: neutral
Molecular weight: in the range of 500,000 daltons to 11,000,000 daltons
Particle size: in the range of 30 μm to 100 μm
Water content: 3.9% (w/w)
Sodium chloride: 4.0 (w/w)
Sodium glycolate: <2.0 (w/w)
Degree of crosslinking: 34%
Crosslinking agent: Na₃P₃O₉ (sodium trimetaphosphate)

Comparative mass spectrometry and IR spectroscopy measurements of Medication 2 and the pharmaceutical composition of the invention (Medication 1) were performed. IR spectroscopy results show that both samples are constituent with carboxymethyl starch sodium salt. The IR spectroscopy analysis did not find any differences between the two samples except for the five signals for trisodium citrate in the sample of the pharmaceutical composition of the invention (Medication 1). Results of the mass spectrometry measurements further confirm that the pharmaceutical composition of the invention (Medication 1) contains sodium citrate while Medication 2 does not. Mass spectrometry did not reveal any further differences as well.

Forty Lewis rats were used for the study. They were housed under standard conditions, had continuous access to fresh water and were fed with standard nutrition. Their well-being was monitored through daily examinations of observed changes during application. In addition, they were weighed on a daily basis so that deviations from normal body weight development could be determined. All protocols with respect to quality of life of the animals were carried out in compliance with national and European laws.

Ten animals were each randomly assigned to one of the four groups:
1. Control group (no adhesion inhibition measures were taken, animals only received sterile 0.9% saline solution)
2. Pharmaceutical composition of the invention (Medication 1)
3. Carboxymethylated starch without sodium citrate (Medication 2)
4. Sodium citrate without carboxymethylated starch (Solution of sodium citrate 3.5 wt. % in sterile 0.9% saline solution, Medication 3)

Ketamine and xylazine were used for the anesthesia. The required level of narcosis was achieved once the flexor-reflexes were suppressed. After shaving, the abdomen was cleaned with ethanol and iodine solutions. Access to the abdomen was achieved by a median laparotomy of approximately 3.5 cm. The induction of adhesions was carried out in accordance with the OPAM. The cecum was exposed and kept moist. The visceral peritoneum of the cecum was then repeatedly rubbed with a watery gauze over an area 1×2 cm in size. A surface of the parietal peritoneum on the right abdominal wall 1×2 cm in size, including the inner muscle layer was sharply resected.

After both defects were created, solutions and starch powders were applied upon them. In group 2 and 3, a total of 300 mg of the powdered carboxymethylated starches were applied per animal and afterwards dripped with 1.2 ml 0.9% isotonic saline solution to turn the powder into an adhesion barrier gel. In group 4, a total of 2.4 ml of the sodium citrate solution was applied per animal.

After application of the respective interventions, the two defects were drawn in spatial proximity to one another using a 4/0 prolene stitch in order to induce maximum adhesions. The abdomen was closed with a dual layer closing technique using a consecutive stitch. The animals were monitored postoperatively until they were fully awakened.

On the seventh postoperative day, the animals were killed during application. In order not to destroy a potentially formed adhesion, the peritoneal cavity was opened through an incision to the left of the original laparotomy scar. During the autopsy, the adhesion formation between the defective abdominal wall and the cecum was macroscopically evaluated. For this purpose, the following system was utilized:

| Score | Description |
|---|---|
| 0 | No adhesions |
| 1 | Thin filmy adhesions |
| 2 | More than one thin adhesion |
| 3 | Thick adhesion with focal point |
| 4 | Thick adhesion with planar attachment |
| 5 | Very thick vascularized adhesions or more than one planar |

Adhesion values are represented as arithmetic averages with standard deviations (SD). Since most of the data sets did not follow a Gaussian distribution (determined with the D'Agostino-Pearson normality test), the multiple comparison of the adhesion assessment was carried out using the Kruskal-Wallis-Test, followed by Dunn's multiple comparison test for non-parametric data (which use the correction for multiple comparisons with the aid of statistical hypothesis testing). Groups were considered as significantly different from one another if p<0.05.

Results

The tested animals all showed a comparable viability and body weight development. One of the animals of group 4 had to be prematurely killed due to complications; the other 39 animals completed the experiment.

Adhesions were scored in the four groups as follows:
Group 1: control (no adhesion inhibition measures, only sterile 0.9% saline solution): 9 of 10 animals showed massive adhesions, each of which received the maximum number of points (five), whereas the tenth animal developed no adhesions at all and therefore received a zero. The arithmetic mean was 4.5 (SD: 1.6).
Group 2: pharmaceutical composition of the invention (Medication 1): None of the animals developed any adhesions, so that all animals were scored with a zero. The arithmetic mean was 0.0 (SD: 0.0).
Group 3: carboxymethylated starch without sodium citrate (Medication 2): Five animals had only mild adhesions, of these two scored a 0.5, two scored a 1.0 and one scored a 1.5. the other five animals had more severe adhesions, of these two scored a 3.0, two scored a 3.5 and one scored a 4.0. The arithmetic mean was 2.2 (SD: 1.4).
Group 4: sodium citrate without carboxymethylated starch (Medication 3): One of the animals had no adhesions and scored a zero, four had mild adhesions, of these, three scored a one and one scored a 2.0. Four had severe adhesions, of these, three scored a 4.0 and one scored a 5.0. One animal did not receive a score as it had to be prematurely terminated due to complications. The arithmetic mean was 2.4 (SD: 1.8).

In comparison to the control, the three treatment groups were able to achieve the following adhesion reductions (calculated as (mean of the control group—mean of the treatment group)/mean of the control group *100):
Group 2: 100%
Group 3: 52%
Group 4: 46%

The statistical comparison using Dunn's multiple comparison as describe above yielded:

| Comparison | p-value | Significant? |
|---|---|---|
| Group 1 vs. group 2 | <0.001 | Yes |
| Group 1 vs. group 3 | 0.128 | No |
| Group 1 vs. group 4 | 0.305 | No |
| Group 2 vs. group 3 | 0.034 | Yes |
| Group 2 vs. group 4 | 0.018 | Yes |
| Group 3 vs. group 4 | >0.999 | No |

Group 2 (Medication 1)) showed a significant reduction in adhesion compared to each of the other three groups. While the other two interventions (carboxymethylated starch without sodium citrate and sodium citrate without carboxymethylated starch) did not show a significant improvement compared to the control group; and were also not significantly different from one another.

The results of this study show that there are hardly any to no postoperative adhesions apparent in previously traumatized mesothelial (or mesothelium-like) surfaces after one week of treatment with the pharmaceutical composition of the invention. Moreover, a complete wound closure and a very good submesothelial healing is apparent.

Example 2: Macroscopical/Histological Proof of Improved Submesothelial Healing with Carboxymethylated Starch Comprising Sodium Citrate

The pharmaceutical composition of the present invention comprises carboxymethylated starch comprising sodium citrate. The pharmaceutical composition of the present invention mixed with saline forms a gel. The so formed gel has several effects:
(1) When applied to mesothelial surfaces, it forms a temporary mechanical barrier so that injured mesothelial surfaces do not adhere to each other.
(2) The sodium citrate contained in the pharmaceutical composition of the present invention inhibits fibrin polymerisation and, thus, prevents the formation of fibrin bridges between the mesothelia, which are the cause of adhesion.
(3) Furthermore, the gel acts as a matrix in which connective tissue healing of tissue defects is promoted.

The efficiency of the pharmaceutical composition of the present invention to prevent adhesion formation was tested in rats utilizing the Optimized Peritoneal Adhesion Model

(OPAM). OPAM technique provides a superior, reproducible induction of severest adhesions of injured areas., i.e. agglutination. Due to the high reproducibility in incidence and extend of adhesion formation, the OPAM is ideal to determine the efficacy of adhesion inhibiting medical devices.

In addition, with OPAM healing processes in tissue defects can be analysed, as in this model a defined abdominal wall defect is created.

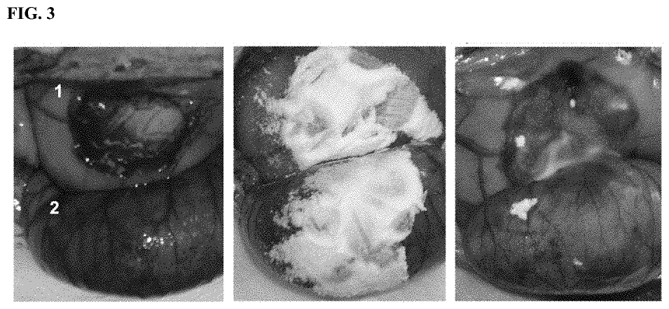

FIG. 3 visualizes the steps of testing a gel comprising the pharmaceutical composition of the present invention to prevent adhesion formation and tissue regeneration using the OPA model.

The left image shows in the upper part the surgical induced abdominal wall defect (1). In the center of the defect, musculus transversus abdomini, musculus obliquus internus abdomini, and musculus obliquus externus abdomini are completely transected. In the lower part (2) the cecum depleted from peritoneum by abrasion with a sponge is visible. The middle image shows the situation after application of 300 mg the pharmaceutical composition of the present invention in powder form into the abdominal wall defect and on the injured cecum. The right image shows the situation after transformation of the powder into gel by application of 4 parts of saline solution per 1 part powder.

For macroscopic and microscopic evaluation, the test animals were sacrificed after one week.

FIG. 4 shows the macroscopic view of the abdominal situs on day 7 in a control animal. Control animals did not receive any adhesion inhibition measures. The cecum is integrated into the abdominal wall. This means that healing of the tissue defect of the abdominal wall in the sense of filling the defect with a scar in the abdominal wall has not occurred.

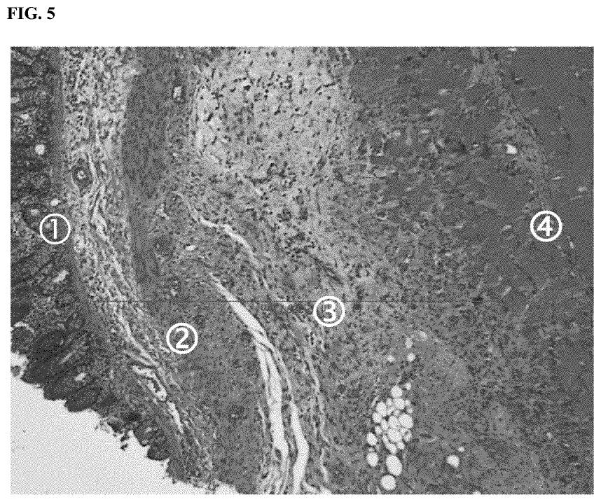

FIG. 5 shows the histology of a control animal at the border of the zone where the cecum has grown into the abdominal wall. One can see how the muscularis of the cecum is closely fused with the striated musculature of the abdominal muscles without separate healing of the abdominal wall defect with a scar.

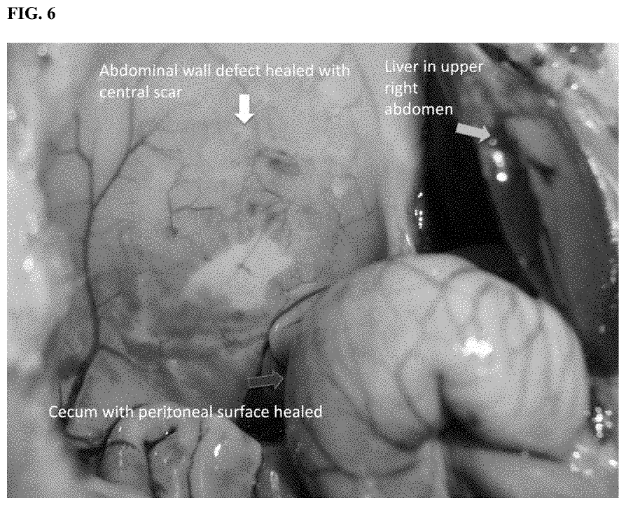

FIG. 6 shows the macroscopic view of the abdominal situs on day 7 in an animal treated with gels made from the pharmaceutical composition of the invention in powder form. The cecum and abdominal wall are not fused and have healed separately. Furthermore, there are no adhesion bridges between the mesothelial surfaces. The peritoneal surface of the cecum and abdominal wall appear macroscopically normal with a reflecting surface.

Furthermore, the abdominal wall defect is filled with a central whitish scar. The thickness of this scar corresponds to the thickness of the normal abdominal wall.

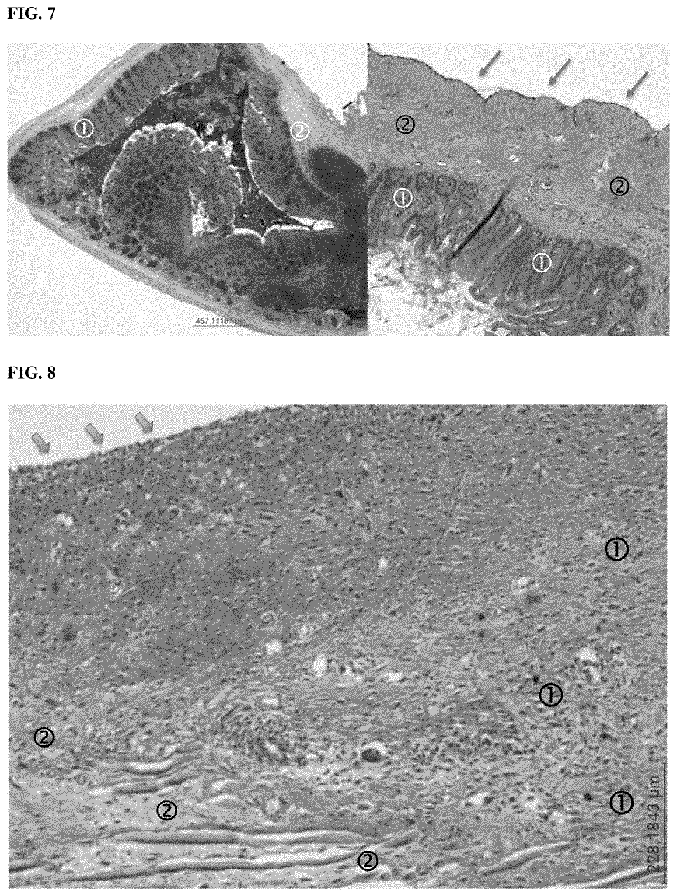

FIG. 7 shows the histology of the cecum of an animal with treatment of gels made from the pharmaceutical composition of the invention in powder form. The right half of the image confirms that the cecum has healed without the formation of adhesions or bands of adhesion. The left half of the image shows that there is complete regeneration of a normal (single layer) peritoneal epithelium.

FIG. 8 shows the histology of a healed abdominal wall defect after 7 days of treatment with a gel made from the pharmaceutical composition of the invention in powder form. The defect is completely filled with fibrous scar. The scar is covered with a layer of normal single layer peritoneal epithelium. In the presence of the gel, a fibrous scar corresponding to the thickness of the abdominal wall forms in the tissue defect of the abdominal wall, whereas without gel the abdominal wall merely adheres to adjacent tissue structures. It can be concluded that the gel made from the pharmaceutical composition of the invention in powder form serves as a matrix for fibrous scar healing of the tissue defect.

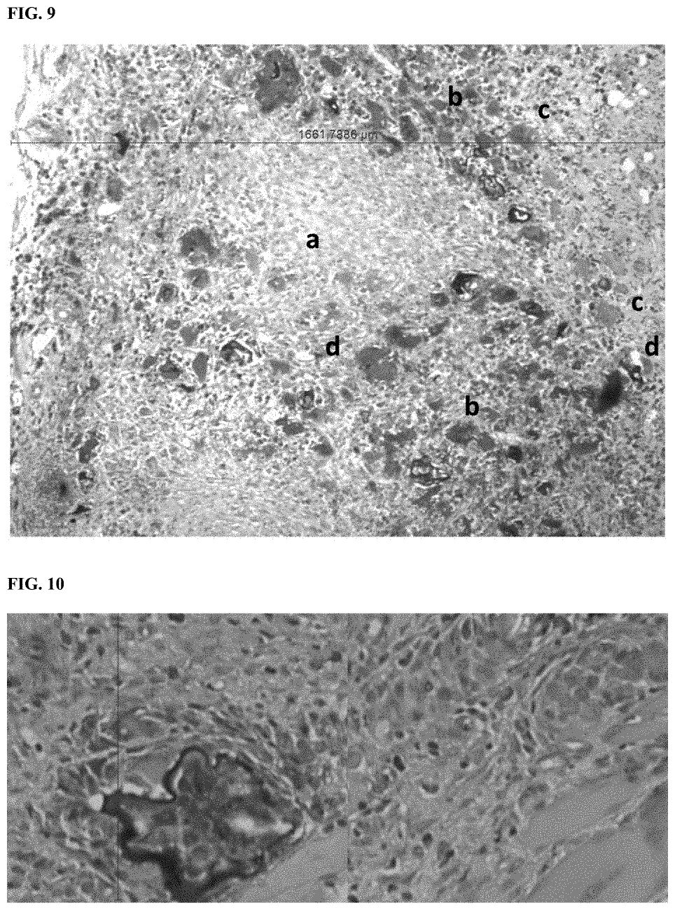

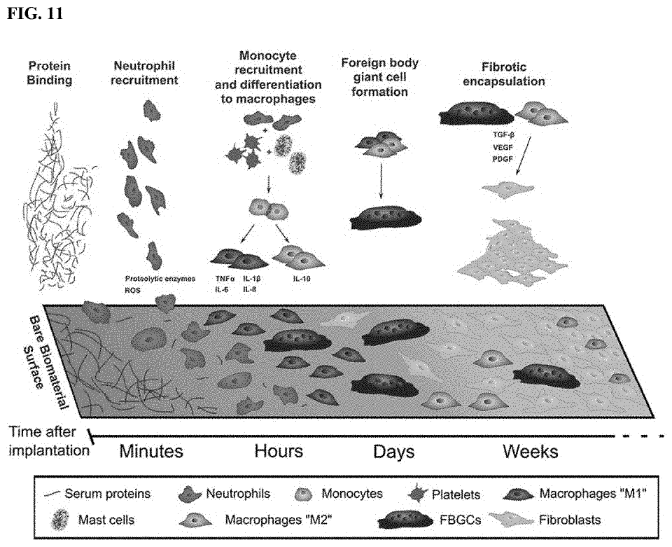

The histological findings of the horizontal sections parallel to the mesothelial surface of the healed abdominal wall also support the function of the gel as a matrix for the healing of the abdominal wall defect (FIG. 9). Here, in the center of the healing defect, there are remnants of particles of the pharmaceutical composition of the invention surrounded by macrophages or containing macrophages, fibroblasts and emerging connective tissue, giant cells formed from fused macrophages, newly formed small vessels and capillaries.

FIG. 10 shows the final process steps of the resorption of hydrobeads of the pharmaceutical composition of the invention and peritoneal wall healing.

Histological findings within the healing abdominal wall defect showing remnants (residues) of hydrobeads, macrophages and giant cells, fibroblasts, and emerging connective tissue indicate that submesothelial healing after one week is improved due to application of said gel.

1. A pharmaceutical composition comprising a carboxymethylated starch and 1 to 20 percent by weight of a compound selected from the group consisting of citric acid, a salt of citric acid, and a mixture thereof, relative to the weight of the carboxymethylated starch.

2. The pharmaceutical composition of claim 1, wherein said pharmaceutical composition comprises a carboxymethylated starch and 1 to 20 percent by weight of a salt of citric acid relative to the weight of the carboxymethylated starch, wherein said salt of citric acid has a solubility in water of greater than 100 g/l, preferably greater than 200 g/l, more preferably greater than 300 g/l, most preferably greater than 400 g/l, at a temperature of 25° C.

3. The pharmaceutical composition of claim 1, wherein said salt of citric acid is selected from the group consisting of a sodium citrate, a lithium citrate and a potassium citrate.

4. The pharmaceutical composition of claim 1, wherein said salt of citric acid is trisodium citrate.

5. The pharmaceutical composition of claim 1, wherein said pharmaceutical composition is present in powder form or in the form of a gel.

6. The pharmaceutical composition of claim 1, wherein said pharmaceutical composition further includes water.

7. The method of claim 1, wherein said carboxymethylated starch is present as a sodium salt of a carboxymethyl ether of the starch.

8. The pharmaceutical composition of claim 1, wherein said carboxymethylated starch has a molecular weight in the range of 500,000 daltons to 11,000,000 daltons.

9. The pharmaceutical composition of claim 1, wherein the starch particles of said carboxymethylated starch have a particle diameter in the range of 30 μm to 100 μm.

10. The pharmaceutical composition of claim 1, wherein said salt of citric acid is on the surface of carboxymethylated starch particles.

11. The pharmaceutical composition of claim 1, wherein said pharmaceutical composition comprises carboxymethylated starch and 1 to 20 percent by weight trisodium citrate relative to the weight of the carboxymethylated starch, and water, wherein said carboxymethylated starch has a molecular weight in the range of 500,000 daltons to 11,000,000 daltons, and wherein particles of the carboxymethylated starch have a particle diameter of 30 microns to 100 microns.

12. The pharmaceutical composition of claim 11, wherein said trisodium citrate is on the surface of carboxymethylated starch particles.

13. The pharmaceutical composition of claim 1, wherein particles of said carboxymethylated starch are made up of about 4.5×10¹⁰ to 2.3×10¹² amylose molecules and 5.6×10⁷ to 1.3×10¹⁰ amylopectin molecules.

14. The pharmaceutical composition of claim 1, wherein carboxymethylated starch is crosslinked having a degree of crosslinking in the range of 25% to 45%.

15. The pharmaceutical composition of claim 1, wherein said carboxymethylated starch has a pH value in solution in the range of 3.0 and 7.5.

16. The pharmaceutical composition of claim 1, wherein said pharmaceutical composition is an aqueous gel comprising crosslinked carboxymethylated starch, citrate ions and water, wherein the citrate ions are present in a concentration in the range of 1 mmole/liter to 500 mmoles/liter of the water, and wherein the crosslinked carboxymethylated starch has a degree of substitution in the range of 0.2 to 0.4 and a degree of crosslinking in the range of 25% to 45%.

17. A method for inhibiting postoperative adhesions, said method comprising administering to a subject in need thereof a pharmaceutical composition according to claim 1.

18. The method of claim 17 comprising the following steps:
i) Providing the pharmaceutical composition according to claim 1 as a powder;
ii) Administering the powder of step i) directly on a postoperative area or site in a subject; and
iii) Forming a gel of carboxymethylated starch covering the postoperative area or site
By residual body fluids present at the postoperative area or site; or
By drizzling the powder after administration on the postoperative area or site with an aqueous liquid,
Wherein said gel of carboxymethylated starch comprises citric acid and/or a dissolved salt of citric acid.

19. The method of claim 17, said method comprising the following steps:
i) Providing the pharmaceutical composition according to claim 1 as a powder;
ii) Premixing the powder of step i) with an aqueous liquid in an amount sufficient for a gel;
iii) Forming a gel; and
iv) Administering the gel of step iii) on the postoperative site in a subject.

20. The method of claim 17, said method comprising the following steps:
i) Providing the pharmaceutical composition as a gel; and
ii) Administering the gel of step i) on the postoperative area or site in a subject.

21. The method of claim 19, wherein said aqueous liquid is selected from the group consisting of water, isotonic saline solution, or hypotonic, isotonic, or hypertonic saline solution as the liquid phase, which comprises one or more cations selected from the group 2 0 consisting of sodium, potassium, ammonium, magnesium, calcium, iron(II), iron(III), aluminum; and/or further comprising one or more anions selected from the group consisting of fluoride, chloride, bromide, iodide, oxide, sulfide, carbonate, sulfate, phosphate, nitrate, chromate, permanganate, and hexacyanoferrate(II).

22. The method of claim 19, wherein said aqueous liquid is selected from the group consisting of Ringer's solution, Ringer's acetate solution, and Ringer's lactate.
